(19) United States
(12) Patent Application Publication
(10) Pub. No.: US 2008/0177150 A1
(43) Pub. Date: Jul. 24, 2008

(54) Molecular Sensors for Bio-Metric Measurements and Bio-Assays

(76) Inventors: Mutsuya Ii, Seattle, WA (US); Jan Schiefer, Seattle, WA (US); Glenn R. Engel, Snohomish, WA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/624,591

(22) Filed: Jan. 18, 2007

(51) Int. Cl. A61B 5/00

(52) U.S. Cl. 600/300

(57) ABSTRACT

A sensor system, for performing measurement and diagnostic testing within a body comprises a sensor, for sensing a characteristic of the interior of a body under study, the sensor including a passive identification tag. The system further comprises a communication apparatus, to be disposed in proximity to the body, for communicating with the sensor to obtain information on the characteristic.

### BACKGROUND OF THE INVENTION

The invention pertains to the field of nanotechnology. The invention has applicability to the field of biometrics, including but not limited to applications such as use of time difference of arrival molecular sensors, for biometric location measurement and bio-assays.

In many different fields of research and measurement, various types of information are to be obtained from within an enclosed body. In the field of biometrics, for instance, the enclosed body might be a human body or other living organism. In other contexts, an enclosed vessel may have limitations on possible means of access to the vessel's interior, etc. Where it is desired to obtain certain types of desired information, the ability to obtain such information may be limited by the degree of invasiveness the body can tolerate. In biometrics, in particular, surgery or toxic foreign substances may be able to obtain such information, but at an unacceptable cost in terms of harm or inconvenience to the human patient.

X-rays have been used to determine and define structural information. Contrast dyes have been used in conjunction with X-ray and CT Scanning and Magnetic Resonance Imaging, to produce high resolution and definition of biological variations. However, although magnetic resonance has not shown adverse effect on patients, large X-ray doses have been shown to be detrimental. This has limited the applicability of such techniques, particularly for patients with particularly sensitive conditions, such as pregnant women.

### SUMMARY OF THE INVENTION

A sensor system, for performing measurement and diagnostic testing within a body comprises a sensor, for sensing a characteristic of the interior of a body under study, the sensor including a passive identification tag. The system further comprises a communication apparatus, to be disposed in proximity to the body, for communicating with the sensor to obtain information on the characteristic.

Further features and advantages of the present invention, as well as the structure and operation of preferred embodiments of the present invention, are described in detail below with reference to the accompanying exemplary drawings.

### BRIEF DESCRIPTION OF THE DRAWINGS

FIG. 1 is a schematic diagram of a system of sensors and communication apparatus in an embodiment of the invention.

FIG. 2 is a more detailed schematic diagram of a sensor embodying the invention.

### DETAILED DESCRIPTION

Structural information, biochemical functions, and tracking of physical movement are of interest in biometric applications. New techniques for obtaining such biometric information, and performing such functions, improve such applications. In particular, biometric apparatus implemented in nanotechnology facilitate such biometric applications measurement and diagnostic techniques.

Embodiments of the present invention include a combination of several aspects which heretofore have been used, if at all, independently. New applications arise as a result of the combination. Here is a brief summary of the above-referenced aspects, which are combined within embodiments of the invention:

First, the use of passive identification tags such as Radio Frequency Identification (RFID) has been in existence for quite some time. RFID tags, at macroscopic sizes with dimensions of the order of centimeters, are commonly placed, for instance, on retail consumer products to deter theft. Notwithstanding the inference that the name “Radio Frequency” implies a limitation on the range of frequencies, within the electromagnetic spectrum, employed by RFID systems, it is in fact the case that many different frequencies are used. For instance, low-frequency tags operate at around 125 KHz, high-frequency (13.56 MHz) and ultra-high-frequency or UHF (860-960 MHz) and Microwave (2.45 GHz).

Second, nanotechnology is an ever, increasing area of popular research. Sub-micron lithography is currently possible and valuable, for fabricating nanotechnology structures using, for instance, semiconductor fabrication techniques. More recently, the capability to create atomic level device structures has emerged.

Third, position locating systems have employed various techniques for determining the position, within a region of three-dimensional space, of a remote object. One such technique is called Time Difference Of Arrival (TDOA). An example of a TDOA system is the Long Range Navigation (LORAN) system used for navigation. Sub-nanosecond timing accuracy by means of pulse-stretching has also been in use within oscilloscopes, vector signal analyzers and other measurement equipment, to accurately determine when the onset of the burst of energy has occurred in relation to a sample clock (another term for this is “Low Time Jitter IF Triggering”).

Embodiments of the present invention combine the first and third above-referenced capabilities to provide systems for obtaining location information of minute (that is, small) sensors, which can potentially be used within the human body. Alternatively, such sensors may be introduced within other vessels, etc., depending on the particular type of sensor information which is desired. Such sensors may be implemented in nanotechnology, as per the second above-referenced element.

A sensor embodying the invention may be microscopic in size, to facilitate entry into the human body, etc., in a variety of ways depending on the particular use to which they will be put, such as by swallowing, injection into the bloodstream or other body fluids, etc.

In an embodiment, such a sensor may be a passive component, which is not actively powered, but rather causes a response when affected by an external stimulus. For instance, responsive to an external RF field, the sensor may generate a digital identification sequence (ID), as is the case with RFID tags. When so activated, the sensor will return a unique ID differentiating it from other similar sensors, allowing each sensor to be located independently.

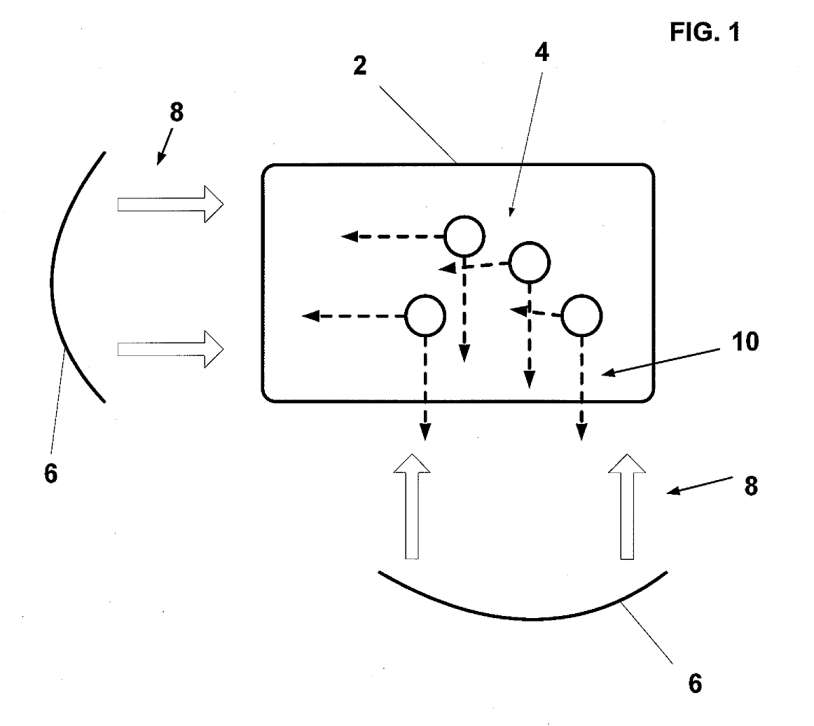

FIG. 1 is a schematic illustration of sensors embodying the invention, at use. In a body 2, such as a human body, vessel, etc., various sensors 4 have been introduced. An external communication apparatus 6, located in suitable proximity to the body 2, generates a stimulus 8, such as an RF field, which reaches the sensors 4 and stimulates them to generate a response. The response, shown as response signals 10, may be the above-mentioned ID, etc. As shown, the communication apparatus 6 includes multiple transceivers, which are positioned at various locations surrounding the body 2, so that the response signals 10 received by the different transceivers may be correlated, to triangulate the positions of the sensors 4. The correlation may, for instance, include receiving the response signals 10 in the form of complex (i.e., real plus imaginary) data which is correlated to determine the time difference of arrival (TDOA).

In an embodiment, the sensors 4 may be implemented including semiconductor integrated circuits (“ICs” or “chips”). Where the response signals 10 are electromagnetic signals generally of a given wavelength, the ICs of the sensors 4 may be fabricated to include antennas (not shown) that are suitable for producing such response signals 10.

The communication apparatus 6 receives the response signals 10, and interprets them to obtain the information that is desired. For instance, if it is desired to locate the site to which the sensors 4 have migrated (such as a tumor or organ of interest), the location of the sensors may be determined, for instance by the above-mentioned TDOA technique. Alternatively, the sensors 4 may be able to sense biometric or diagnostic information, such as temperature, the presence of a chemical substance of interest, etc., and provide response signals 10 representative of such diagnostic information.

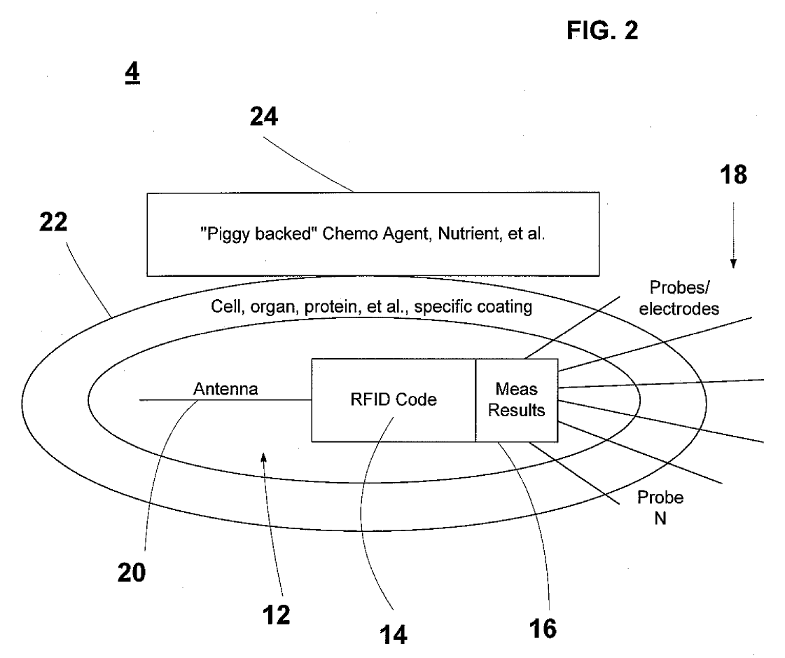

FIG. 2 is a more detailed diagram of one of the sensors 4. The sensor 4 includes a sensing and communicating apparatus 12, which may be implemented electronically, for instance as a semiconductor IC chip. The apparatus 12 includes an ID tag such as an RFID code 14, and a processor 16 for measuring and determining results based on sensing information obtained through probes 18. The apparatus also includes an antenna 20, for receiving and sending communications via electrical signals. The sensor 4 additionally includes an exterior coating 22, which may be customized for specific compatibility or affinity, or the lack thereof, with a particular cell, organ, cellular matrix protein, other protein, etc., to be found within the body within which the sensor 4 will operate. In addition to the coating 22, there may also be a “piggy-backed” chemotherapy agent, other chemical agent, nutrient, medication, etc., to be released after the sensor 4 migrates to a desired site, such as an organ, tumor, etc.

Mobility of the sensors occurs through the natural processes of the body, organism, or other vessel under analysis. In some applications, it may be desirable for sensors, after being introduced, to migrate to a particular site or substance of interest. Such sensors may be modified or customized to facilitate attraction (or lack thereof) to the site or substance of interest, such as to various components, substances, organs, tumors, cell proteins, etc.

This may be achieved through customizing the housing (or coating) of the sensor itself. For instance, such sensor housings or coatings could be designed to have affinities for various types of cellular matrix proteins (as in the case of measuring sizes of cancer tumors), attraction to certain compounds, etc. or simply to track the rate of blood flow, or of digestive or assimilation processes, within the body. For example, various incarnations of fibronectin, an extracellular adhesion molecule, could be used to provide such attraction to provide cellular specificity.

In an embodiment, the sensors may be removed or neutralized without undue invasiveness. This may be achieved, for instance, if the sensors themselves are passed through the body and excreted. Alternatively, the sensors may be formulated to have bio-degradation attributes such that, over time, the sensors themselves would dissolve and pass through the body.

In general, there is a relation between the frequency of the response signals 10 and the required power; that is, higher frequency response signals 10 require higher power. However, such higher frequency response signals 10 may be better able to pass through higher density components and fluids, such as intervening body tissues, etc.

There is also a general relation between the physical size of the sensors 4 and their distance from the communication apparatus 6 This relation is generally due to the size of the antenna etched onto the chip of the sensor 4. For instance, a sensor 4 that is the size of a grain of pepper may need to be within an inch of the communication apparatus 6, for the stimulus signals 8 to reach the sensors 4 and/or for the response signals 10 to reach the communication apparatus 6.

Signal strength as a function of the high frequencies necessary for microscopic wavelengths typically result in the need for extremely close proximity of the receiver of the stimulated signal. However, with the use of correlation techniques, such as those described in co-pending U.S. patent application 2006-0250264, Cutler et al., “Method and System for Computing and Displaying Location Information from Cross-Correlation Data” for TDOA, this requirement may be mitigated substantially. With these cross-correlation techniques, signals buried within the noise floor can nevertheless be extracted and provide good distance measurements.

Where a group of sensors are introduced within a body and their locations are to be determined, they may be synchronized with respect to a single sample clock, each employing pulse-stretching technology to align signal arrival time to the necessary accuracy (spatial resolution) for a given application. The system notes the time of arrival for each signal, and determines the distance to the sensor with respect to time of arrival or through the use of correlation methods. The exact position, within or around the body, of each of the sensors is determined by receiving the RFID signal. These determined positions may be used to establish a reference frame, which may be used for the intended biometric, bio-assay, or diagnostic purpose.

In another embodiment of the system of the invention, there may also be employed one or more sensors on the surface or exterior of the body, at known locations. For instance, in medical applications such sensors may be provided in adhesive, paste-on appliques, similar to probes conventionally used for electrocardiograms, etc. Such external sensors may be used for correlation, etc., to facilitate or enhance the location of the sensors within the interior of the body.

Registration of the body position with respect to the sensors may be through pressure transducers, or by means of light sensitive sensors below the body with illumination cast from above.

Variations of this microscopic “sensor” could be used to measure attributes as well as provide its unique code to be used for spatial location within a given structure. For instance, when binding or adhesion has occurred with the molecular receptors, a code could be appended indicating this fact. Similarly, using the externally provided RF energy to create a potential difference within points within the sensor a small current could be applied to measure characteristics as in conductivity. This could clearly be extended in a number of ways for a variety of physical parameters.

The specificity of the receptors on these sensors could also provide the possibility of “piggy-backing” chemotherapy agents on the surface of the sensors, to perform therapeutic tasks such as to destroy cancer cells - reducing exposure of bio-toxins to the body overall. Inversely, nutrients, drugs or “repair-agents” could be specifically targeted to cells with deficiencies. Combining the ability to determine successful adhesion with the delivery of the therapy agent provides additional security in delivery to the targeted cells or structures.

A system embodying the invention may be employed for motion or rate of flow analysis. For instance, where blood flow is slow or obstructed, a system embodying the invention may be used to measure the blood flow and identify the locations where such slow flow or obstruction is occurring. This may be done by taking multiple location measurements, for instance at specified time intervals, and observing the change of position of individual sensors. Alternatively, Doppler compensation may be employed to derive magnitude and direction of motion from the received signals.

Additionally, it is possible to use equipment such as a dialysis machine to extract the RFID tags based on their ID signals. This would allow the user to selectively extract cells or other samples from the body that were tagged with specific IDs. Determination of which tags to extract may be a result of the analyses described above.

Surgical procedures may also employ sensors embodying the invention. For instance, sensors might be customized to have adhesive, magnetic or other properties that could be used to group and pull abnormal cells together and away from normal cells. This would facilitate surgical extraction of tumors with more exacting precision.

While much of the preceding discussion has focused on embodiments in which the sensors 4 receive an outside stimulus signal and transmit their IDs, sensed information, etc., in response to that stimulus signal, in other embodiments the sensors 4 may perform such transmission without the outside stimulus, but rather according to other criteria. For instance, the sensor 4 might transmit every time a specified period of time elapses. Alternatively, the sensor 4 might transmit responsive to a condition within the environment surrounding it. For instance, the sensor 4 might transmit upon encountering a given organ, tumor, protein or other chemical substance, etc., or upon encountering a given fever temperature, pH, etc. Also, the sensor 4 might build up thermal energy, electrical charge, etc., from its environment, and transmit when sufficient such energy has been built up to power it.

While the above-discussed embodiments of the invention are employed in connection with biological entities such as human bodies, other embodiments of the invention may be applied to any subject, vessel, apparatus or medium where mobility can be achieved through existing processes, such as fluid flow, Brownian motion, air flow, etc.

Although the present invention has been described in detail with reference to particular embodiments, persons possessing ordinary skill in the art to which this invention pertains will appreciate that various modifications and enhancements may be made without departing from the spirit and scope of the claims that follow.

What is claimed is:

1. A sensor system for performing measurement and diagnostic testing within a body, the system comprising:
a sensor for sensing a characteristic of the interior of a body under study, the sensor including a passive identification tag; and
a communication apparatus, to be disposed in proximity to the body for communicating with the sensor to obtain information on the characteristic.

2. A sensor system as recited in claim 1, wherein the sensor responds to a stimulus signal by producing a response signal.

3. A sensor system as recited in claim 1, wherein the sensor includes a semiconductor integrated circuit chip.

4. A sensor system as recited in claim 3, wherein (i) the sensor responds to a stimulus signal by producing a response signal, and (ii) the semiconductor integrated circuit chip includes an antenna for transmitting the response signal.

5. A sensor system as recited in claim 2, wherein the response signal includes an ID that is unique to the sensor.

6. A sensor system as recited in claim 2, wherein:
the sensor obtains one of one of (i) biometric information, (ii) bio-assay information, and (iii) diagnostic information, from its surroundings in the body; and
the response signal further includes the information obtained by the sensor.

7. A sensor system as recited in claim 1, wherein the sensor is customized to facilitate migration, within the body, to one of (i) a site, and (ii) a substance, of interest.

8. A sensor system as recited in claim 7, wherein the sensor includes a customized housing.

9. A sensor system as recited in claim 8, wherein the customized housing includes a chemotherapy agent.

10. A sensor system as recited in claim 8, wherein the customized housing includes a substance having an affinity for a predetermined cellular matrix protein.

11. A sensor system as recited in claim 1, wherein the communication apparatus includes (i) a transmitter for transmitting an electromagnetic signal to the sensor; and (ii) a receiver for receiving an electromagnetic response signal from the sensor.

12. A sensor system as recited in claim 1, wherein:
the sensor system further comprises multiple sensors implemented in nanotechnology for sensing a characteristic of the interior of the body under study, each of the multiple sensors including a respective passive identification tag unique to that sensor; and
the communication apparatus includes a receiver (i) for receiving respective response signals from the multiple sensors, each respective response signal containing the passive identification tag that is unique to the respective one of the multiple sensors, and (ii) for identifying respective ones of the multiple sensors based on the respective passive

13. A sensor system as recited in claim 1, wherein the sensor is microscopic in size.

14. A sensor system as recited in claim 1, wherein the passive identification tag of the sensor includes an RF (radiofrequency) ID tag.

15. A sensor system as recited in claim 1, wherein the communication apparatus includes multiple receivers, to be disposed at respective locations around the proximity of the body, for receiving the signals from the sensor, and triangulating the position of the sensor based on the received signals.

16. A sensor system as recited in claim 1, wherein the communication system further includes a Doppler compensation apparatus for deriving magnitude and direction of motion of the sensor from the received signals.

17. A sensor system as recited in claim 1, wherein the sensor sends signals responsive to a condition within the environment surrounding the sensor.

18. A sensor system as recited in claim 1, wherein the sensor is implemented in nanotechnology.
